(19) United States
(12) Patent Application Publication
(10) Pub. No.: US 2024/0115257 A1
(43) Pub. Date: Apr. 11, 2024

(54) Integrated Closure Device

(71) Applicant: Robert Lee Bromley, El Paso, TX (US)

(72) Inventor: Robert Lee Bromley, El Paso, TX (US)

(21) Appl. No.: 17/803,652

(22) Filed: Oct. 11, 2022

(51) Int. Cl. A61B 17/04

(52) U.S. Cl. CPC A61B 17/0469; A61B 17/0482

(57) ABSTRACT

A novel integrated wound closure device for laparoscopy having proximal activation means, a suture staging and management means, a distal anchoring means, an integrated tissue piercing means, a suture placement and suture grasping means, in an elongated body which can be reused multiple times per patient.

### BACKGROUND OF INVENTION

Prior art involves many methods of laparoscopic wound closure. For many years a small suture guide device having at least two guide channels, but not limited to two guide channels, originating from a large proximal end, and exiting at an angle on a tapered shaft. Said device was inserted into the surgical wound created by a trocar. When the trocar is removed the wound will need to be closed with suture. Said small device has a tapered shaft compatible for the size of the trocar used. Said device is inserted into the wound until said large proximal end is in contact with the abdomin which sets the correct depth of said device. The surgeon will use a small grasper having a means to hold suture and a sharp distal end to pierce the patient tissue. Said grasper will hold suture as it is inserted into a first guide channel. Said guide channel aides in placing suture at the correct position to properly pierce the peritoneum. After properly piercing said peritoneum and placing said suture the surgeon will release the suture and withdraw said grasper. Using a second guide channel said grasper is inserted and the grasper is oriented to capture the dangling suture inside the body cavity previously placed. Upon capture of said suture said surgeon will pull said suture back through said second guide channel and withdraw said grasper. Said suture is of sufficient length to have suture ends exposed and available for said surgeon to tie said suture and close the wound. Said suture guide had a single purpose of guiding suture placement, made as a single component usually of plastic or stainless steel. There were no moving parts or additional functions in this prior art.

This embodiment will be concerned with devices that have at least two or more combined functions used for wound closure. For those skilled in the art, it is well understood that single function devices known as guides are used in cooperation with a separate and non-integral suture grasper having a sharp distal end for piercing tissue and grasping suture.

A popular prior art wound closure device is comprised of two distinct components: an elongated body in the shape of a shaft having internal acute angled guide channels with a distal end having an anchoring means involving pivoting members that contract and expand when the proximal end is operated from a natural first position to a second position and back again. Said proximal end is usually spring loaded to return said proximal end back to a first natural position. The purpose of said pivoting members prevent removal of said wound closure device from within the body cavity by the geometry change of said anchoring means usually forming a triangular shape when expanded and a cylindrical shape matching said elongated body when contracted.

An external and separate suture grasping device has a piercing distal end commonly constructed to have a suture grasping means. Said piercing distal end can be constructed in a means such as a distal end jaw configuration that opens and closes to hold or release suture. Another prior art suture grasper has a distal piercing point similar to a hypodermic needle having stored inside a dual end flexible metal wire to facilitate retracting or extending from said distal piercing point. Said internal metal wire may have different end configurations designed to capture and release suture. Said suture grasping device is external of said wound closure device and enters the body through said acute angled guide channels.

An additional prior art method involves the above configuration but having two curved needles integral to said anchoring means attached at the distal end of the elongated shaft. Said curved needles have suture affixed to them. Said curved needles are held in place at said anchoring means and the excess suture length between said needles is placed in a groove retaining the loop of suture along said elongated body. Said anchoring means is slimmed to allow insertion into the patient wound. Said curved needles are sized so the arc of the needle is confined within the diameter of said elongated shaft and penetrate the peritoneum upward from the distal end of said wound closure device through the peritoneum. As the curved needles complete their circular travel they are stored within said elongated body for removal.

The embodiment of this novel integrated wound closure invention combining multiple functions into a single elongated device with a proximal selective means to operate said multiple functions. The details of which will be clearly itemized in the drawing descriptions.

### SUMMARY OF THE INVENTION

A novel invention for a laparoscopic wound closure device comprising: a means to stage suture in a pre-determined target location, an operable means to sequentially engage functions necessary to close a surgical wound and maintain surgical position for inserting integrated sharp needles through the peritoneum and up to the target location, extending a grasper from said sharp needles to capture and retract targeted suture within said sharp needles. Upon wound withdrawal of said wound closure device suture ends are exposed so the surgeon may tie a knot and close the wound.

### BRIEF DESCRIPTION OF DRAWINGS

FIG. 1 is an overall view of the systems included in this novel invention.

FIG. 2 is an exploded view of the components comprised therein said novel invention.

FIG. 3 is an isometric view of the novel invention showing the main body halves.

FIG. 4 is an exploded view of the proximally located selector system.

FIG. 5 shows the close-up view of the distally located anchoring system.

FIG. 6 shows the overall anchoring system components.

FIG. 7 shows the perspective view of the integrated internal needle piercing system.

FIG. 8 shows the perspective view of the integrated suture capturing system.

FIG. 9 shows a perspective view of initiating suture contact.

FIG. 10 shows a perspective view of suture being engaged.

FIG. 11 shows a perspective view of suture being captured and retracted.

FIG. 12 is an overall view of the suture routing path as staged for a wound closure procedure.

### DETAILED DESCRIPTION OF DRAWINGS

The following drawings will depict and illustrate one means of construction for this novel integrated closure device but those skilled in the art can readily understand that the construction shown is used to portray the functional details of this embodiment and not necessarily reflect the actual shape and assembly of production components.

Said novel invention called an integrated closure device 100 combines five distinct systems, 300, 400, 500, 600, 700 into said device 100 which are operable by the surgeon during wound closure. Each system will be described in detail after the overall description of said device 100. Said device 100 facilitates suture loading by a sterile field technician using off the shelf suture and staging said suture onto said device 100. The proximal end has a selective system 300 for a first downward movement which extends and closes the distal anchoring system 400, and a suture management system 500 for routing and holding suture. After loading suture, the sterile field technician will then hand-off said device 100 to a surgeon to insert said device 100 into the wound. The second downward movement of the selective system 300 opens the anchoring system 400 to allow contact with the peritoneum setting the correct surgical depth. The beginning portion of the third downward movement of the selective system 300 extends the piercing system 600 out the guide channels 120 through the peritoneum until reaching a stop, the remaining portion of the third downward movement extends the grasper system 700 beyond said piercing system 600 to capture the suture at the target location. Upon release of said proximal end selective system 300 a spring returns said grasper system 700 into said piercing system 600. A grasper hook 712 is drawn inside the sharp distal end 626 until finally the sharp distal end 626 returns to the stowed position in the distal channel 120. The fourth downward movement of the proximal selective system 300 closes the anchoring system 400 and the device 100 is withdrawn from the bringing the suture ends out of the wound. Said suture ends slip out of the retaining ring 510 as said device 100 is moved away from the wound. Said, free suture ends are then tied by the surgeon to close the wound.

Said invention has a first system means called a selector system 300 which selectively operates all three functions 400, 600, 700, in an orderly manner by rotating 60 degrees on each full stroke down and up. Said second system means called an anchoring system 400 prevents the removal of said device 100 from the patient peritoneum. Said third system means called a piercing system 600 has sharp distal ends 626 for piercing the peritoneum and retracting from said peritoneum. Said fourth system means called a grasping system 700 is a flexible wire hook 712 which extends beyond said sharp distal ends 612 to reach over said suture 530 to retrieve said suture 530 at the target location span 424 and retracting said suture 530 into said sharp distal end 612.

Said device 100 is first operated by said sterile technician. To begin, said device 100 is removed from a sterile pouch. Said device is in a normal state with said anchoring system 400 open. The first operation is to depress the selective system 300 plunger button 310 which rotates a shaft 330 in 60-degree increments for each downward movement. As said plunger button 310 is depressed the plunger 330 having a ramp 334 causes rotation by 30 degrees upon contact with a distal ramp 354. Upon release of the plunger button 310 the plunger ramp 336 strikes a lead surface 347 or 348 to cause an additional 30-degree rotation, placing said plunger ramp 336 against a fence 346 to hold the anchoring system 400 closed. The sterile technician will acquire some suture from a storage cabinet and remove it from the holder and if equipped will detach a needle. Said sterile technician holding the suture ends in one hand whereby two vertical strands forming a loop when draped. Said device 100 having a trough 434 on said distal nose 430 is placed against the suture loop allowing said suture to nest in said trough 434 of distal nose 430. Said sterile technician routes one suture strand into the lower wing 420 grooves 422 straddling an open window span 424, around and upward into the upper wing 410 grooves 412 straddling an open window span 414, then under the distal nose 112, feeding one strand into the suture track 114 and suture guide 118 to be finally pulled into a pinch gap 515 for suture end retention. The suture 530 follows said grooves 412, 422 as they trace a staging path near the pivot points 232, 412, 426, 428, 432 to maintain a near zero length change when the anchoring system 400 is open or closed. The alternate suture strand is staged in an identical fashion albeit on the opposite lower wing 421, upper wing 411, tab 113, suture track 115, and finally pulled into a pinch gap 515. With the anchoring system 400 retained in the closed condition and suture secured by the pinch gap 515, said device 100 with staged suture 530 is handed to the surgeon.

During a laparoscopic surgical procedure said device 100 is used to close surgical wounds created by laparoscopic trocar devices. If the trocar is an 8 mm trocar it will leave a wound slightly larger than 8 mm; therefore, said integrated closure device 100 will be sized to correspond to the wound diameter whether 8 mm by this example or some other size. The surgeon usually removes the trocar from the wound and then inserts the integrated closure device 100 to begin the process of closing a wound. Said integrated closure device can be reloaded and reused multiple times per patient.

Since suture 530 used in this novel invention 100 is pre-loaded in the staged position by a sterile field technician the surgeon carefully plunges said device 100 into the patient wound until reaching the desired depth. The surgeon depresses the plunger button 310 causing selector tabs 332 to abut proximal driver ends 452 protruding through a slot 382 in said collar 380 downward as the shaft 330 rotates 30 degrees at the end of stroke. Upon release of the plunger button 310 a spring 450 returns said anchoring system 400 to a triangular shape whereby the wings 410 are pulled into contact with the peritoneum. Said anchoring system 400 is held in the normally open position by the spring 450.

After said anchoring system 400 was opened said piercing system 600 is ready to pierce said peritoneum. Depressing said selector plunger button 310 allows the plunger fins 332 to push a shelf 384 on the non-rotatable selector collar 380 downward. The piercing system 600 includes said pusher 640 having proximally attached needle shafts 622, flex joint 624, and sharp distal end 626. Said sharp distal end 626 is retained in said distal guide channels 120, 220 having a bend 122, 222 and straight run 124, 224. Said sharp distal end 626 is stored within the distal guide channels 120, 220. Said sharp distal end 626 has a hypodermic style pierce point 612 being parallel to the elongated body axis 116 so as not to expose the sharp distal end 626 when retracted. Said selector collar 380 having two 60 degrees of collar slot 382 for operating said anchoring system 400, and two 120 degrees of collar shelf 384 for operating piercing system 600 and grasper system 700. The first 60 degrees of said 120 degrees of collar shelf 384 activates the piercing and grasping systems 600, 700 respectively. When the selector fins 332 contact the collar shelf 384 said beginning stroke drives the collar 380 and subsequently drives the needles 626 through the peritoneum passed the open window span 414 of the upper wing 410 and said collar 380 hits a beginning stroke stop 132. Depressing the selector button 310 after the stroke stop 132 compresses the needle spring 650 extending the grasper system 700 hook 712. Said grasper system 700 having a distal hook 712 on an angular leg 714 allows said hook 712 to pass over said staged suture 530 spanning said open window 422 until said suture touches said angular leg 714. Upon reversing said ending stroke said hook 712 captures said suture 530 and retracts said suture 530 into said sharp distal end 626. For suture to retract into said sharp distal end 612 of said novel invention 100 the additional suture length is drawn from the suture ends slipping through the pinching gap 512 as tension is created by a pinch ring 510 and pinch spring 520.

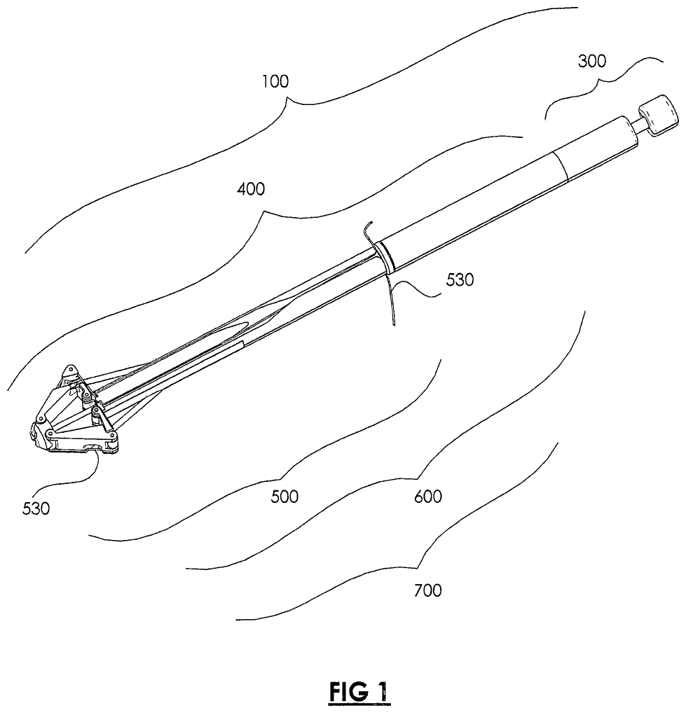

FIG. 1 is an overall view of the functional systems, 300, 400, 500, 600, 700, integrated in this novel invention 100 comprised of a proximal selective function means 300, a distal anchoring means 400, suture management system 500, and internal needle piercing means 600, and a suture grasping means 700.

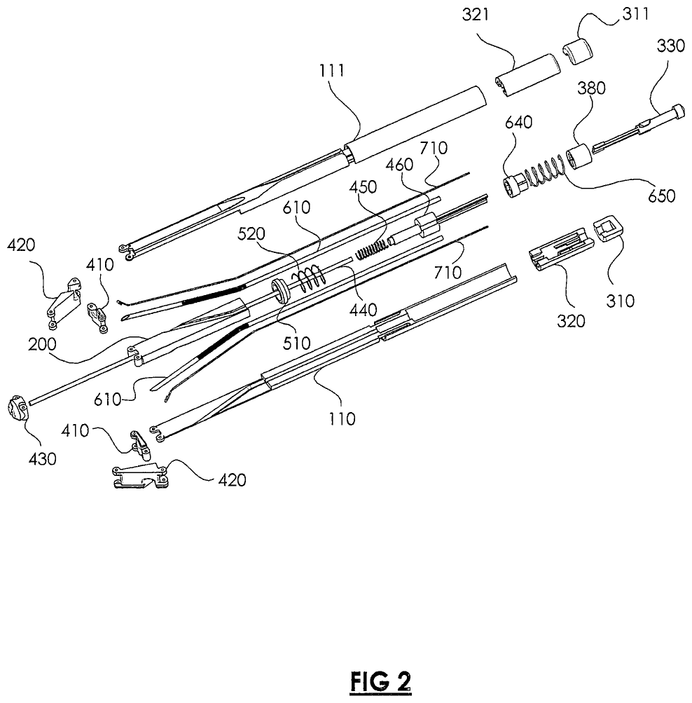

FIG. 2 is an exploded view of the components comprised therein of said novel invention showing said elongated body 110, 111, the central distal end 200, the proximal selective function means 300. Said proximal selective function means 300 is comprised of a shaft 330, a vertically retained but freely rotating button halves 310, 311. Said shaft 330 is constrained within the two indexing halves 320, 321 which on their interior surfaces have features that allow the shaft 330 to rotate 360 degrees in 60-degree increments from 30-degrees down to 30-degrees up. The shaft 330 contacts the driver 460 by passing through the gaps 382 of the collar 380 to operate the anchor system 400. The shaft 330 can also push collar 380 by shelf 384 to move the piercing system 600 via the spring 650 contacting the pusher 640. The spring 650 has sufficient force to cause the pusher 640 to move until stopping against the driver 460 proximal end. Upon stopping the shaft 330 continues to depress the spring 650 until the grasper system 700 extends beyond the sharp distal end 612 capturing the suture. Releasing the button 310,311 allows the shaft 330 to return due to the spring 650. Finally, depressing the shaft 330 allows the contact of the driver legs 462, 464 to move the driver 460 and compress the spring 450 opening the anchoring system 400.

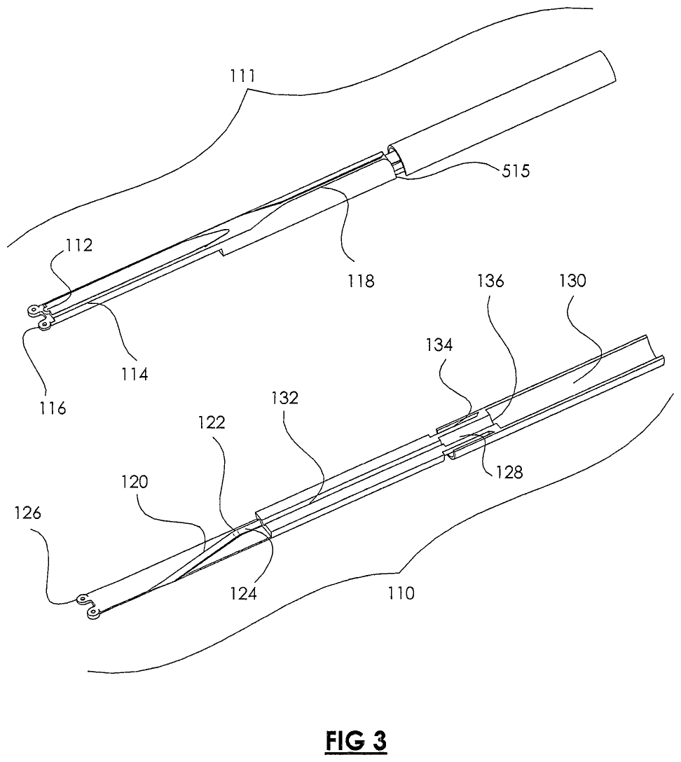

FIG. 3 is an isometric view of said novel invention showing the main body halves 110,111. The body halves 110, 111 are functionally symmetrical. Said upper half 111, shows exterior features such as the distal nose 112 between the hinge means 116, 126 respectively shown as knuckles using a hinge pin but those skilled in the art can imagine the hinge means being a living hinge or snap-in knuckle without a pin, and the suture track 114 leading to the suture guide 118 ending at the suture pinch gap 515. Said lower half 110 shows the interior features of a guide channel 120, a bend 122, and straight channel 124 which in combination guides and aligns the piercing system means 600 to pierce the peritoneum. There is a center channel 132, a spring recess 134, a spring pocket 128, a shoulder 136 and main cavity 130.

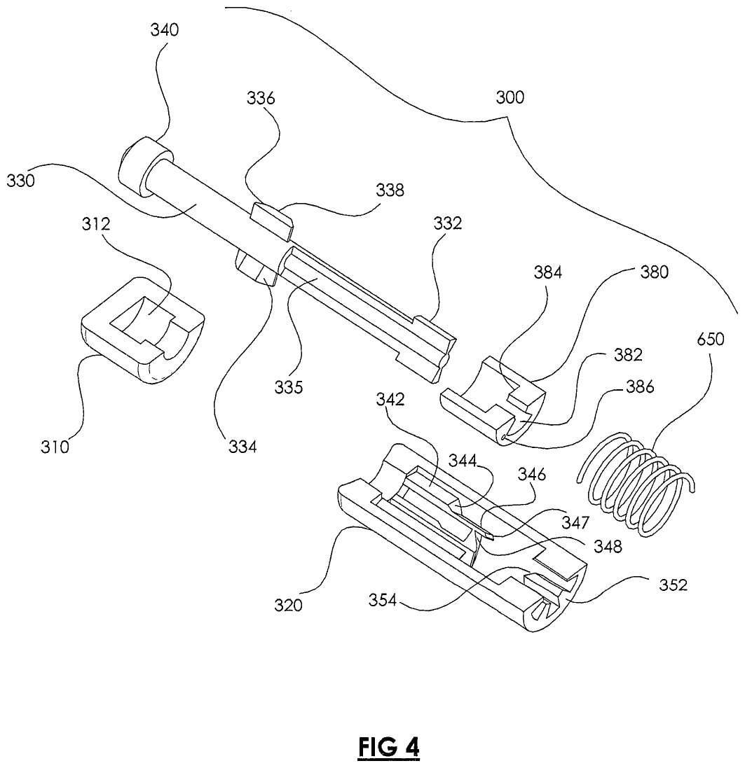

FIG. 4 is an exploded view of the selector system means 300 showing a split button 310 for clarity having an interior cavity 312 to encapsulate vertically but not limit rotation of the shaft head 340. The shaft 330 having two opposing distal tabs 332 emanating from the shaft axis forming a pie shape 30 degrees in size, which selectively operate functions in one of three segments, each 60-degrees, by incrementing 30 degrees in the downward direction and 30 degrees in the upward direction before repeating. Each distal tab 332 rotates 180 degrees after three operations. A first operation begins by depressing the shaft 330 via the button 310. Near the mid-point of the shaft 330 are two opposing facing fins 338 emanating from the shaft axis forming a pie shape at 30 degrees in size, on the same plane as said distal tabs 332. The fins 338 having a trapezoidal shape due to distal ramps 334 and proximal ramps 336. Interior of the housing 320 is shown split for clarity have a total of six proximal lugs 342 and six distal lugs 352, said lugs are offset by 60 degrees so as not to be above one another. Each of said lugs 342, 352 have ramps. Proximal lugs 342 have distal ramps 344 adjacent to a fence 346 and fence ramp 347, while six distal lugs 352 have proximal ramps 354 which interact with said fins 334 ramps. Fin ramps 336 interact with proximal lug ramps 344 and fence ramps 347. Each downward interaction rotates said shaft 330 clockwise by 30 degrees and each upward movement caused by spring 650 rotates said shaft 330 clockwise by an additional 30 degrees. Collar 380 has 60-degree bow tie shaped opening 382, to allow the shaft fins 332 to pass through and compress the spring 450. The collar 380 has a solid shelf 384, between said bow tie shaped opening 382, of approximately 120 degrees which is pushed downward by the shaft 330 tabs 332 when compressing said spring 650. Not compressing the spring 650 allows the shaft 330 to open and close the anchor system, compressing the spring 650 allows the shaft to drive said needle piercing system 600 and said suture grasping system 700 sequentially.

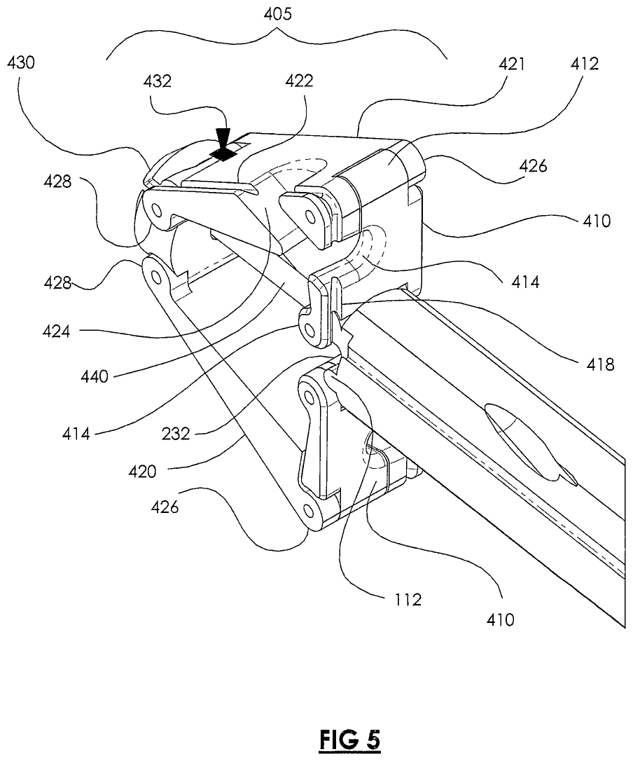

FIG. 5 shows the perspective view close-up of the anchoring system 405 distal end in the open triangular shaped position. In the open position the upper wing 410 is perpendicular to the longitudinal axis of said device 100 while the lower wing is at an acute angle to said longitudinal axis. The peritoneum surface will make intimate contact with the upper wing 410 to set the proper position of this novel invention 100 in respect to the peritoneum. The anchoring system 400 is comprised of hinge points 232, 412, 426, 428, 432, shown as knuckles and pins, but not limited to knuckles and pins, allowing the following components to pivot starting distally: distal end cap 430 with a hinge means 432 and is fixedly attached to the drive rod 440; lower wing 420, 421 with a hinge means 426, 428; upper wing 410, 411 with a hinge means 412, 414; and center distal end 200 with a hinge means 230. When the anchor system 405 is closed the drive rod 440 pushes downward and the lower wings 420, 421 and upper wings 410, 411 become nearly parallel to said drive rod 440.

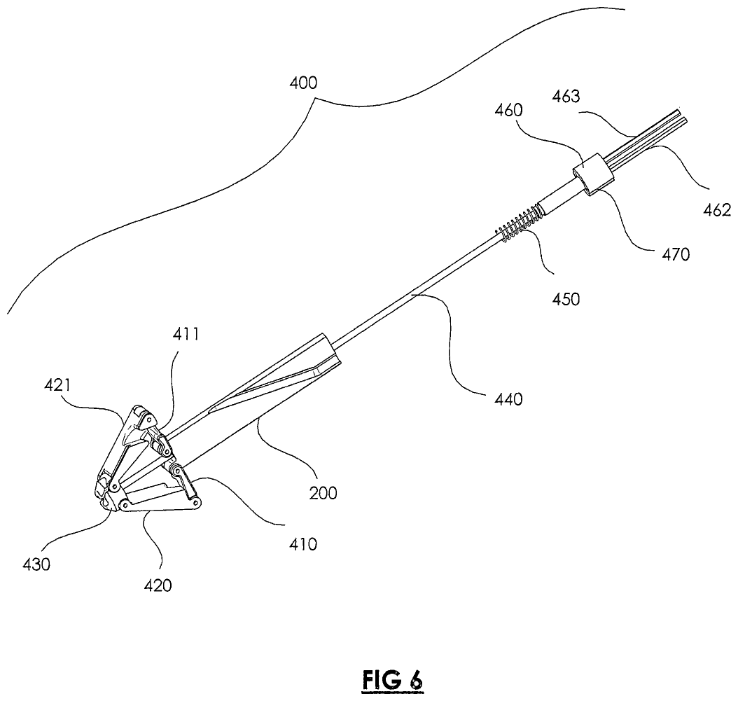

FIG. 6 is an isometric view of the anchoring system 400 having a proximal end that interacts with the selector system 300. The proximal end has a pusher 460 that is fixedly attached to the proximal end of the rod 440. Said pusher 460 has two proximal legs 462, 463 and mates with a spring 450 to return the distal end anchor system 405 to a normally open triangular position as shown. When the rod 440 is pulled by the spring 450 the end cap 430 moves toward the proximal end and the pivot points allow the anchor system 405 to expand into a triangular shape or when pushed distally by the selective system 300 contract to a near parallel position to allow insertion into the surgical wound. The central distal end 200 attaches to the upper wings 410, 411 pivot points and guides the rod 440 to move along the elongated axis.

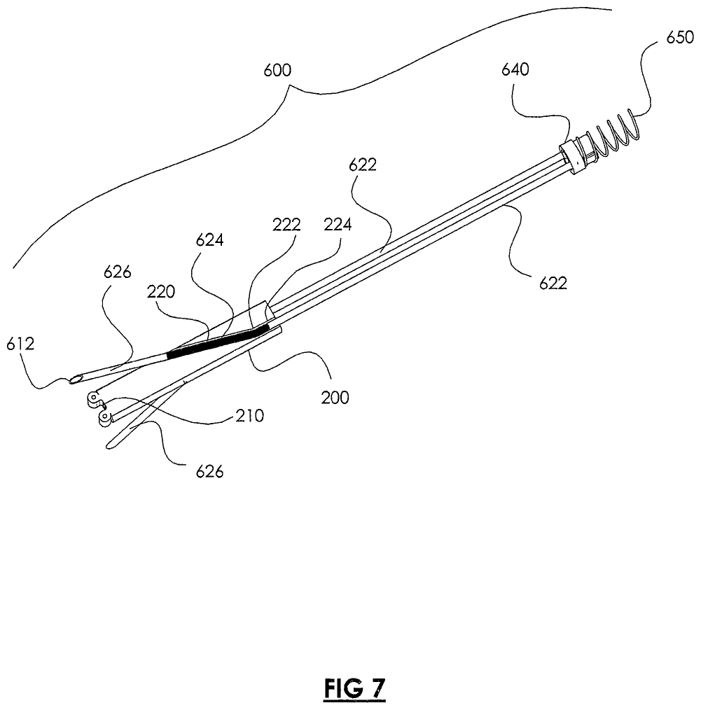

FIG. 7 shows the perspective view of the needle piercing system 600. Said needle piercing system 600 is comprised of a short distal end 626 having a sharp hollow point 612, similar but not limited to, a hypodermic needle with the opposite end fixedly attached to a flexible member 624 having near zero compression and near zero elongation properties. Said flexible member 624 is depicted as an extension spring, but not limited to a spring, as other means are available such as, but not limited to, semi-rigid plastics, dead soft malleable metals, memory metal, spring metal strip or spring wire to allow movement as it passes through the bend 222 and straight channel 224. Said flexible member 624 proximal end is attached to a straight hollow tube 622 that is fixedly attached to the pusher 340 which is in contact with a spring 650. Central distal end 200 having an acute angle guide channel 220 to reliably pierce the peritoneum at a sufficient distance from the tissue edge to allow said suture to close said wound without tearing the peritoneum. The peritoneum wall is perpendicular to the elongated axis of said novel invention 100 at the distal flat end 210 of the central distal end 200.

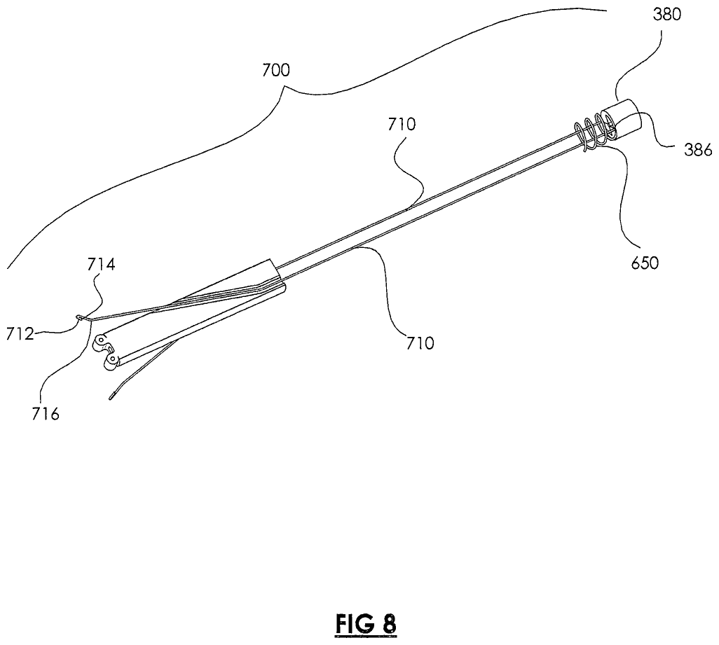

FIG. 8 shows the perspective view of the suture capturing system 700 having a flexible wire 710 with the proximal end means to firmly attach at the desired location 386 of the collar 380 to resist separation from pulling or pushing. A spring 650 controls when the suture capturing system 700 may extend or retract. The flexible wire 710 having a distal end hook 712 held at an angle by the bent leg 714 at a correct angle for the distal end hook 712 to intersect said suture for capture.

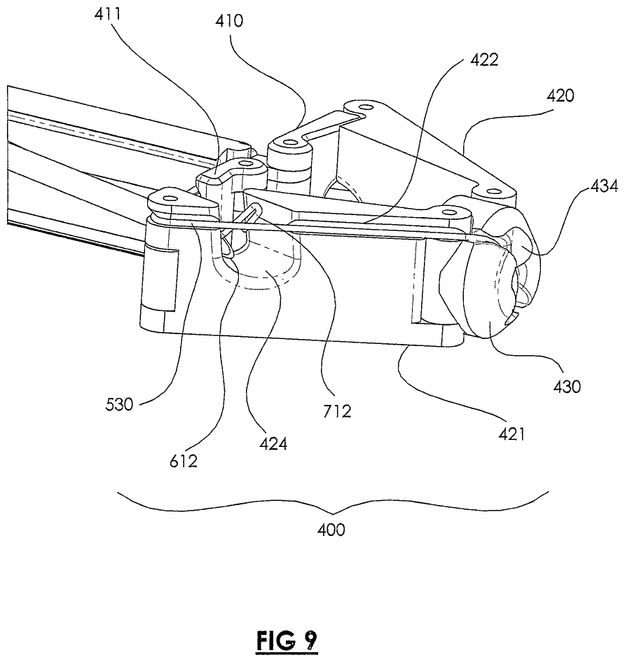

FIG. 9 shows a perspective view of initiating suture contact by extending the hook 712 beyond the sharp distal end 612. Due to the orientation and position of the suture 530 held in the groove 422 across the span 424 the suture 530 lies at the mid contact point of the hook 712. Said lower wing 420 and upper wing 410 have aligned span openings 424, 414 respectively as illustrated by the triangle shape of the anchor means 400. As described earlier when said suture 530 is held in the trough 434 of the end cap 430. Said trough 432 aligns the suture 530 to follow the grooves 424 in the lower wing 420, 421 and groove 414 in the upper wing 410, 411.

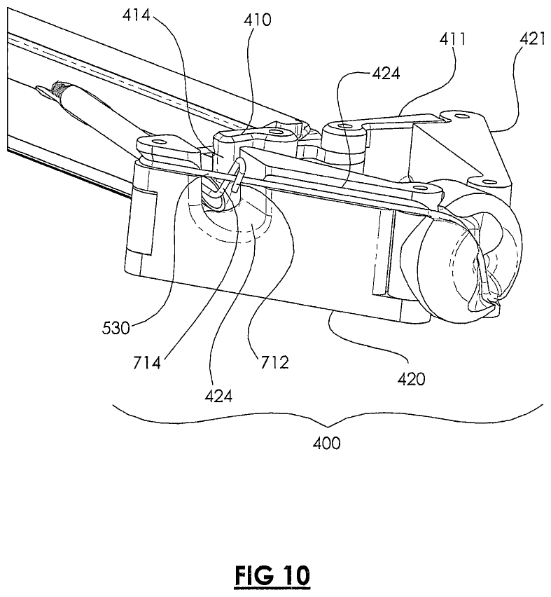

FIG. 10 shows a perspective view of the suture staging means 500 as the suture 530 becomes engaged by the suture grasping means 700 hook 712 formed in a U shape at an acute angle of 5 degrees, but not limited to 5 degrees, held at an angle by the leg 714. Said long wire 716 has a bend to form an angled leg 714 supporting said hook 712. The leg 714 allows the hook 712 to pass over said suture 530 being held in place by the anchor groove 422 of the lower wing 420, 421. After the hook 712 passes through the span 424 extending beyond the suture 530, the suture 530 will by force of the angle to slip under the hook 712 and against the angled leg 714 whereby the suture returns to its' original plane along the groove 422 as shown.

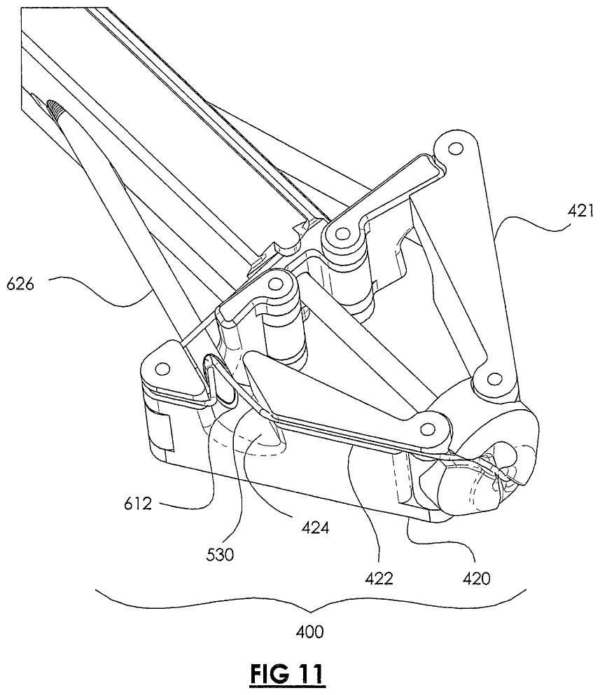

FIG. 11 shows a perspective view of suture 530 captured and retracted into the hollow shape of the needle 626 by the retracted suture grasper hook 712. The suture 530 is held in place by the groove 422 of the lower wing 420, 421. As the suture 530 is pulled into the captured position and the needle 626 also retracts into said device 100 the length of suture 530 required for said movement is released by said suture management system 500 further detailed in FIG. 11.

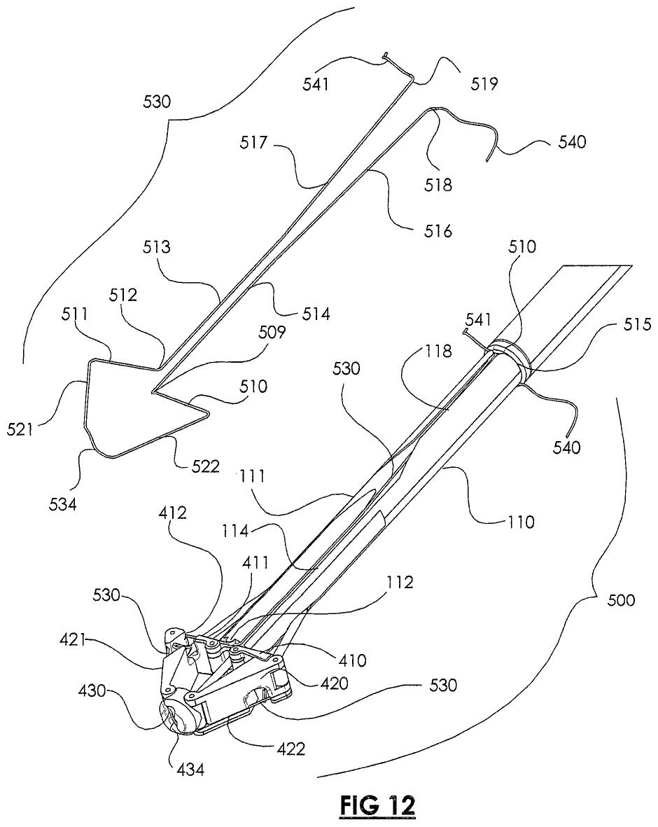

FIG. 12 is an overall view of the suture management system 500 as staged for a wound closure procedure. The upper view shows the suture 530 in the staged configuration without visual obstruction by the novel invention 100 components. Suture 530 has describable shapes related to said device 100 counterparts. Starting at the distal end cap trough 434 corresponds to the suture trough 534; the lower wing groove 421, 422 corresponds to the 521, 522 path; the upper wing groove 411, 412 corresponds to the 510, 511 leg; the distal tab 112 corresponds to the tab bend 512; the suture track 114 guides the 513, 514 run; the suture guide 118 controls the 517, 518 running up to the retaining ring 510 pinch gap 515 which holds at suture 518, 519 leaving excess suture tails 540, 541 dangling.

The lower view shows the suture 530 in relation to the novel invention 100 from the distal end cap 430 trough 432, passing from the lower wing 420, 421 groove 422 across the target location span 424 into the upper wing 410, 411 groove 412 across the span 414, under the distal tab 112, through the suture track 114 into the suture guide 118 and under the suture ring 510 pinching the suture tails 530 by the pinch gap 515.

What is claimed:

1. An integrated closure device suitable for closing laparoscopic wounds comprising:
an elongated shaft having at least one channel to act as a guide,
a single, but not limited to single, proximal activation means,
said proximal activation means operates multiple functions,
said functions are sequential but not limited to sequential operation,
distal pivoting members to expand or contract in shape,
said pivoting members having a suture staging means,
said proximal activation means contracts said pivoting members for wound entry introducing suture into the wound,
said proximal activation means expands said pivoting members preventing wound egress while holding suture,
at least one integrated distally sharp means stored inside said elongated shaft,
said proximal activation means extends said distally sharp means to pierce said tissue,
said integrated grasping means extends to capture suture at a targeted location,
said proximal activation means returns said grasping means capturing said suture,
said proximal activation means retracts said captured suture into said distally sharp means,
said proximal activation means returns said distally sharp means inside said elongated shaft with captured suture,
said proximal activation means contracts said pivoting means for wound egress,
said suture is pulled through said wound to be knot tied by a surgeon.

2. As in claim 1 whereby said integrated closure device incorporates a slidable disk along said elongated body to adjust for abdominal thickness of the patient keeping said integrated closure device in vertically stable position.

3. As in claim 1 whereby said integrated closure device retains suture ends by friction to allow excess suture to be drawn during capture.

4. As in claim 1 whereby said activation means has at least one proximal activation means for each individual function allowing non-sequential operation.

5. As in claim 1 whereby said device can be reloaded with suture and re-used multiple times per patient.

6. As in claim 1 whereby said device has suture pre-loaded and placed in sterile packaging.

7. As in claim 1 whereby said device can have suture loaded after removal from sterile packaging.
